(19) United States
(12) Patent Application Publication
(10) Pub. No.: US 2008/0027409 A1
(43) Pub. Date: Jan. 31, 2008

(54) Patient hydration/fluid administration system and method

(76) Inventors: Robert I. Rudko, Holliston, MA (US); Mark R. Tauscher, Medfield, MA (US); Andrew Halpert, Brookline, MA (US)

Correspondence Address: Iandiorio and Teska, 260 Bear Hill Rd, Waltham, MA, 02451, US

(21) Appl. No.: 11/823,654

(22) Filed: Jun. 28, 2007

Related U.S. Application Data
(63) Continuation-in-part of application No. 10/936,945, filed on Sep. 9, 2004.

(51) Int. Cl. A61M 5/168

(52) U.S. Cl. 604/503

(57) ABSTRACT

A patient hydration system with a first infusion subsystem for infusing a patient with fluid from a first source and a second infusion subsystem for infusing a patient with fluid from a second source. A urine output measurement subsystem determines the amount of urine output by the patient. A controller is responsive to the first infusion subsystem, the second infusion subsystem, and the urine output measurement subsystem and is configured to control the first infusion subsystem based on the amount of urine output by the patient and/or the amount of infused fluid measured by the second infusion subsystem.

### RELATED APPLICATIONS

This application is a continuation-in-part application of U.S. patent application Ser. No. 10/936,945, filed Sep. 9, 2004, entitled “Patient Hydration System and Method”. This application is also related to co-pending applications Ser. Nos. 11/408,851; 11/408,391; 11/409,171; and 11/580,354 all of which are incorporated herein by this reference.

### FIELD OF THE INVENTION

This invention relates to a patient hydration system and method.

### BACKGROUND OF THE INVENTION

The “cath lab” in a hospital is where a patient is injected with a radiocontrast media, imaged, diagnosed, and often operated on. Typically, a cardiologist refers the patient to the cath lab and the patient is instructed not to eat or drink the night before. In the case of a patient suffering a heart attack, the patient may be transferred directly to the cath lab.

Often, the patient is dehydrated when the patient arrives at the cath lab. The patient is prepped and the radiocontrast media injected. If, after diagnostic imaging, a possible problem is detected, intervention occurs in the form of angioplasty or the placement of a stent. During these procedures, additional radiocontrast media may be injected into the patient and the patient imaged so the interventional cardiologist or radiologist can view the progress of the operation.

Unfortunately, the radiocontrast media can be toxic to the patient especially a patient who is dehydrated at the time the radiocontrast media is injected. A patient who already suffers from various medical problems such as diabetes or kidney problems is even more prone to medical problems due to the injection of the radiocontrast media.

It has been observed that dehydration increases the risk of radiocontrast nephropathy (RCN) when radiocontrast agents are injected into a patient during coronary and peripheral vascular catheterization procedures. RCN is the third most common cause of hospital-acquired renal failure. It occurs in over 5% of patients with any baseline renal insufficiency and can occur in 50% of patients with preexisting chronic renal insufficiency and diabetes. Radiocontrast media has a variety of physiologic effects believed to contribute to the development of RCN. One of the main contributors is renal medullary ischemia, which results from a severe, radiocontrast-induced reduction in renal/intrarenal blood flow and oxygen delivery. The medullary ischemia induces ischemia and/or death of the metabolically active areas of the medulla responsible for urine formation, called the renal tubules. Medullary ischemia is attributed to the increase of oxygen demand by the kidney struggling to remove the radiocontrast media from blood plasma and excrete it from the body at the same time as the normal process of controlling the concentration of urine. Oxygen consumption in the medulla of the kidney is directly related to the work of concentrating urine. Since the presence of radiocontrast media in the urine makes it much more difficult for the kidney to concentrate urine, the work of the medulla outstrips the available oxygen supply and leads to medullary ischemia.

Although the exact mechanisms of RCN remain unknown, it has been consistently observed that patients with high urine output are less vulnerable to contrast injury. It is also clear that dehydration increases the risk of RCN, likely because urine (and contrast media inside the kidney) is excessively concentrated. As a result, patients predisposed to RCN are hydrated via intravenous infusion of normal saline before, during and after the angiographic procedure. Hydration is commonly performed at a conservative rate, especially in patients with existing heart and kidney dysfunction, since over-hydration can result in pulmonary edema (fluid in the lungs), shortness of breath, the need for intubation, and even death. Thus, the patients at highest risk for RCN are those least likely to receive the only proven therapy for preventing RCN (I.V. hydration) due to the unpredictability of side effects from I.V. hydration.

A major limitation to the more widespread use of the already known therapeutic, or optimal, levels of I.V. hydration is the current inability to balance the amount of fluid going into the patient to the amount of fluid being removed or excreted from the patient. It is possible to have a nurse measure a patient's urine output frequently but this method is impractical as nurses are often responsible for the care of many patients. In addition, the only accurate method of measuring urine output is to place a catheter into the patient's urinary bladder. Without a catheter, the patient must excrete the urine that may have been stored in the bladder for several hours. During this time, the amount of I.V. hydration can be significantly less than the amount of urine produced by the kidneys and stored in the bladder, leading to dehydration. Since many patients do not normally have such a catheter during procedures using radiocontrast media, a valid measurement of urine output is not possible.

There seems to be indisputable scientific evidence that RCN in patients with even mild baseline renal insufficiency can lead to long term complications and even increased risk of mortality. This scientific knowledge has not yet been extended to daily clinical practice as routine monitoring of renal function post-catheterization is not usually performed and limits the identification of the known short-term clinical complications.

At the same time, there is a great deal of awareness in clinical practice that patients with serious renal insufficiency (serum creatinine (Cr)≧2.0) often suffer serious and immediate damage from contrast. Many cardiologists go considerable length to protect these patients including slow, overnight hydration (an extra admission day), administration of marginally effective but expensive drugs, staging the procedure or even not performing procedures at all.

There are approximately 1 million inpatient and 2 million outpatient angiography and angioplasty procedures performed in the U.S. per year (based on 2001 data). Based on the largest and most representative published studies of RCN available to us (such as Mayo Clinic PCI registry of 7,586 patients) we believe that 4% of patients have serious renal insufficiency (Cr≧2.0). This results in the initial market potential of 40 to 120 thousand cases per year from interventional cardiology alone. There is also a significant potential contribution from peripheral vascular procedures, CT scans and biventricular pacemaker leads placement. As the awareness of the RCN increases, the market can be expected to increase to 15% or more of all cases involving contrast.

According to the prior art, hydration therapy is given intravenously (I.V.) when someone is losing necessary fluids at a rate faster than they are retaining fluids. By giving the hydration therapy with an I.V., the patient receives the necessary fluids much faster than by drinking them. Also, dehydration can be heightened by hyperemesis (vomiting), therefore the I.V. method eliminates the need to take fluids orally. An anesthetized or sedated patient may not be able to drink. Hydration is used in clinical environments such as surgery, ICU, cathlab, oncology center and many others. At this time, hydration therapy is performed using inflatable pressure bags and/or I.V. pumps. A number of I.V. pumps on the market are designed for rapid infusion of fluids (as opposed to slow I.V. drug delivery) for perioperative hydration during surgery, ICU use and even emergency use for fluid resuscitation.

An infusion pump is a device used in a health care facility to pump fluids into a patient in a controlled manner. The device may use a piston pump, a roller pump, or a peristaltic pump and may be powered electrically or mechanically. The device may also operate using a constant force to propel the fluid through a narrow tube, which determines the flow rate. The device may include means to detect a fault condition, such as air in, or blockage of, the infusion line and to activate an alarm.

An example of a device for rapid infusion of fluids is the Infusion Dynamics (Plymouth Meeting, Pa.) Power Infuser. The Power Infuser uses two alternating syringes as a pumping engine. Since it is only intended to deliver fluids (not medication), the Power Infuser has accuracy of 15%. It provides a convenient way to deliver colloid as well as crystalloid for hydration during the perioperative period among other possible clinical settings. The Power Infuser provides anesthesiologists with the ability to infuse at rates similar to that seen with pressure bags, but with more exact volume control. The maximum infusion rate is 6 L/hr. It has the flexibility of infusing fluid at 0.2, 1, 2, 4 and 6 L/hr. A bolus setting of 250 mL will deliver that volume in 2.5 min. In a large blood loss surgical case, the use of Power Infuser enables large volumes of colloid to be delivered to restore hemodynamics.

It is also known in the art that loop diuretics such as Lasix (furosemide) reduce sodium reabsorption and consequentially reduce oxygen consumption of the kidney. They also reduce concentration of contrast agents in the urine-collecting cavities of the kidney. They induce diuresis (e.g., patient produces large quantities of very dilute urine) and help remove contrast out of the kidney faster. Theoretically, they should be the first line of defense against RCN. In fact, they were used to prevent RCN based on this assumption until clinical evidence suggested that they were actually deleterious. More recently, doubts have been raised regarding the validity of those negative clinical studies.

In two clinical studies by Solomon R., Werner C, Mann D. et al. “Effects of saline, mannitol, and furosemide to prevent acute decreases in renal function induced by radiocontrast agents”, N Engl J Med, 1994; 331:1416-1420 and by Weinstein J. M., Heyman S., Brezis M. “Potential deleterious effect of furosemide in radiocontrast nephropathy”, Nephron 1992; 62:413-415, as compared with hydration protocol, hydration supplemented with furosemide adversely affected kidney function in high-risk patients given contrast. Weinstein et al. found that furosemide-treated subjects lost 0.7 kg on average, whereas a 1.3-kg weight gain was noted in patients randomized to hydration alone, suggesting that in furosemide-treated subjects the hydration protocol has been insufficient and patients were dehydrated by excessive diuresis.

The clinical problem is simple to understand: diuresis is widely variable and unpredictable but the fluid replacement (hydration) at a constant infusion rate is prescribed in advance. To avoid the risk of pulmonary edema, fluid is typically given conservatively at 1 ml/hr per kg of body weight. The actual effect of diuretic is typically not known for 4 hours (until the sufficient amount of urine is collected and measured) and it is too late and too difficult to correct any imbalance. Meanwhile, patients could be losing fluid at 500 ml/hour while receiving the replacement at only 70 ml/hour. The effects of forced diuresis without balancing are illustrated in the research paper by Wakelkamp et. al. “The Influence of Drug input rate on the development of tolerance to furosemide” Br J. Clin. Pharmacol. 1998; 46: 479-487. In that study, diuresis and natriuresis curves were generated by infusing 10 mg of I.V. furosemide over 10 min to human volunteers. From that paper it can be seen that a patient can lose 1,300 ml of urine within 8 hours following the administration of this potent diuretic. Standard unbalanced I.V. hydration at 75 ml/h will only replace 600 ml in 8 hours. As a result the patient can lose “net” 700 ml of body fluid and become dehydrated. If such patient is vulnerable to renal insult, they can suffer kidney damage.

To illustrate the concept further, the effects of diuretic therapy on RCN were recently again investigated in the PRINCE study by Stevens et al. in “A Prospective Randomized Trial of Prevention Measures in Patients at High Risk for Contrast Nephropathy, Results of the PRINCE. Study” JACC Vol. 33, No. 2, 1999 February 1999:403-11. This study demonstrated that the induction of a forced diuresis while attempting to hold the intravascular volume in a constant state with replacement of urinary losses provided a modest protective benefit against contrast-induced renal injury, and importantly, independent of baseline renal function. This is particularly true if mean urine flow rates were above 150 ml/h. Forced diuresis was induced with intravenous crystalloid, furosemide, and mannitol beginning at the start of angiography.

The PRINCE study showed that, in contrast to the Weinstein study, forced diuresis could be beneficial to RCN patients if the intravascular volume was held in a constant state (no dehydration). Unfortunately, there are currently no practical ways of achieving this in a clinical setting since in response to the diuretic infusion the patient's urine output changes rapidly and unpredictably. In the absence of special equipment, it requires a nurse to calculate urine output every 15-30 minutes and re-adjust the I.V. infusion rate accordingly. While this can be achieved in experimental setting, this method is not possible in current clinical practice where nursing time is very limited and one nurse is often responsible for monitoring the care of up to ten patients. In addition, frequent adjustments and measurements of this kind often result in a human error.

Forced hydration and forced diuresis are known art that has been practiced for a long time using a variety of drugs and equipment. There is a clear clinical need for new methods and devices that will make this therapy accurate, simple to use and safe.

Often, another fluid or fluids besides a hydration fluid such as saline is infused into the patient during therapy. Examples include various drugs or a Ph adjuster such as sodium bicarbonate. The rate of infusion of this fluid is typically set by the nurse who adjusts a valve in the line between an IV needle and the bag of fluid. Or, saline can be provided to the patient from one source for hydration and from another source in an I.V. needle to keep the patient's vein open should a drug need to be administered at a later time.

In such a situation, it can now become more difficult to balance the fluid delivered to the patient with the amount of urine output by the patient since the patient is being infused with fluid from two (and in some cases more than two) sources.

The applicant's co-pending applications directed to a balanced hydration system are incorporated herein by this reference. They are U.S. patent application Ser. No. 10/936,945 filed Sep. 9, 2004 entitled “Patient Hydration System and Method”; U.S. patent application Ser. No. 11/408,851 filed Apr. 21, 2006 entitled “Patient Hydration System With a Redundant Monitoring of Hydration Fluid Infusion”; U.S. patent application Ser. No. 11/408,391 filed Apr. 21, 2006 entitled “Patient Hydration System With Abnormal Condition Sensing”; U.S. patent application Ser. No. 11/409,171 filed Apr. 21, 2006 entitled “Patient Hydration System With Hydration State Detection”; and U.S. patent application Ser. No. 11/580,354 filed Oct. 13, 2006 entitled “Patient Connection System For a Balance Hydration Unit”.

### SUMMARY OF THE INVENTION

It is therefore an object of this invention to provide a patient hydration system and method.

It is a further object of this invention to provide such a system and method which prevents kidney damage in a patient.

It is a further object of this invention to provide such a system and method which protects the patient undergoing a medical procedure, for example, a procedure involving a radiocontrast agent.

It is a further object of this invention to provide such a system and method which incorporates a balancing feature intended to prevent dehydration, overhydration, and to maintain a proper intravascular volume.

It is a further object of this invention to provide a balanced diuresis method which automatically balances fluid loss in the urine.

It is a further object of this invention to provide such a system and method which is accurate, easy to implement, and simple to operate.

It is a further object of this invention to provide such a system and method which is particularly useful in the clinical setting of forced diuresis with drugs known as I.V. loop diuretics.

The subject invention results from the realization that patient dehydration and over hydration in general can be prevented by automatically measuring the urine output of the patient and adjusting the rate of delivery of a hydration fluid from more than one source to the patient to achieve, as necessary, a zero, positive, or negative net fluid balance in the patient.

The subject invention features a patient hydration/fluid administration system. A first infusion subsystem is for infusing a patient with fluid from a first source. A second infusion subsystem is for infusing a patient with fluid from a second source. A urine output measurement subsystem determines the amount of urine output by the patient. A controller is responsive to the first infusion subsystem, the second infusion subsystem, and the urine output measurement subsystem and is configured to control the first infusion subsystem based on the amount of urine output by the patient and/or the amount of infusion fluid measured by the second infusion subsystem.

In one example, the first infusion subsystem includes a pump controlled by the controller for infusing the patient with fluid from the first source. The first infusion subsystem may include a first weighing device for weighing the first source and outputting the weight of the first source to the controller. The urine output measurement subsystem may also include a weighing device for weighing a urine collection chamber connected to the patient and outputting the weight of the urine collection chamber to the controller. Typically, the controller is programmed to control the pump based on the weight of the urine collection chamber.

The second infusion subsystem may also include a weighing device for weighing the second fluid source and outputting the weight of the second fluid source to the controller. In one example, the controller is configured to calculate, based on the weight of the second fluid source, the amount of fluid from the second fluid source infused into the patient and/or the rate of infusion of the fluid from the second source.

The subject invention may also feature a regulator for controlling the infusion rate of the fluid from the second source into the patient. In one example, the regulator includes a valve. In another example, the regulator includes a pump. The controller can be configured to adjust the regulator. For example, the controller may be configured to adjust the regulator based on the amount of urine output by the patient. Or, the controller may be configured to adjust the regulator based on the amount of the first fluid infused into the patient from the first source. When the first infusion subsystem includes a pump controlled by the controller for infusing the patient with fluid from the first source, the controller may control both the pump and the regulator. The controller can be configured to control the pump based on the amount of fluid from the second source infused into the patient.

The subject invention also features a fluid infusion measurement device comprising a housing, a first attachment for suspending the housing, a second attachment for suspending a source of fluid from the housing, a weighing device responsive to the second attachment for weighing the source of fluid infused into a patient, and a processor responsive to the weighing device and configured to calculate as an output the amount of fluid from the source infused based on the weight of the source. The housing may include a display for displaying the output of the processor.

The subject invention also features a patient fluid administration management method. A patient is infused with a hydration fluid. A second fluid is administered to the patient. The amount of the second fluid administered to the patient is measured, the patient's urine output is measured and the amount of hydration fluid infused into the patient is controlled based on the measured urine output.

In one example, the measured amount of the second fluid administered to the patient is displayed. The amount of hydration fluid infused into the patient can be controlled based on the measured amount of the second fluid infused into the patient. Also, the amount of the second fluid administered to the patient can be controlled. The amount of the second fluid administered to the patient may be based on the measured urine output. Also, the amount of the second fluid administered to the patient can be based on the amount of hydration fluid infused into the patient.

One system comprises a first infusion subsystem for infusing a patient with fluid from a first source and a second infusion subsystem for infusing a patient with fluid from a second source. The second infusion subsystem includes a housing, a first attachment for suspending the housing, a second attachment for suspending the second source from the housing, and a weighing device responsive to the second attachment for weighing the second source of fluid infused into the patient. A urine output measurement subsystem determines the amount of urine output by the patient. A controller is responsive to the first infusion subsystem, the second infusion subsystem, and the urine output measurement subsystem and is configured to control the first infusion subsystem based on the amount of urine output by the patient and to calculate the amount of fluid from the second source infused based on the weight of the second source.

### BRIEF DESCRIPTION OF THE SEVERAL VIEWS OF THE DRAWINGS

Other objects, features and advantages will occur to those skilled in the art from the following description of a preferred embodiment and the accompanying drawings, in which:

FIG. 1 is a schematic front view of an example of a patient hydration/fluid administration system in accordance with the subject invention;

FIG. 2 is a block diagram of an example of the fluid infusion measurement device shown in FIG. 1;

FIG. 3 is a block diagram showing several primary components of one embodiment of a patient hydration/fluid administration system in accordance with the subject invention;

FIG. 4 is a flow chart depicting one example of the software associated with the controller of this invention and the method of adjusting the infusion rate based on the amount of urine output by the patient; and

FIG. 5 is a flow chart showing an embodiment of the subject invention wherein the amount of a fluid infused into a patient is calculated.

### DETAILED DESCRIPTION OF THE INVENTION

Aside from the preferred embodiment or embodiments disclosed below, this invention is capable of other embodiments and of being practiced or being carried out in various ways. Thus, it is to be understood that the invention is not limited in its application to the details of construction and the arrangements of components set forth in the following description or illustrated in the drawings. If only one embodiment is described herein, the claims hereof are not to be limited to that embodiment. Moreover, the claims hereof are not to be read restrictively unless there is clear and convincing evidence manifesting a certain exclusion, restriction, or disclaimer.

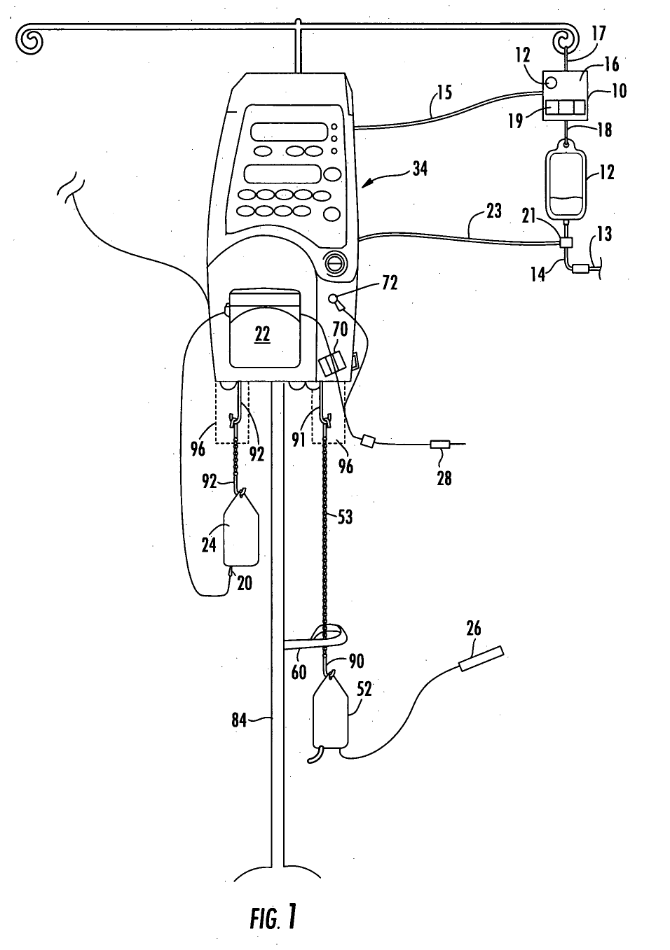

One preferred example of a patient hydration system according to this invention includes unit 34, FIG. 1 typically mounted on IV pole 84. Unit 34 has programmable controller electronics therein. There is an infusion subsystem including pump 22 responsive to source of infusion fluid 24 for infusing a patient with hydration fluid. There is also a urine output measurement subsystem for determining the amount of urine output by the patient. In this particular example, source of infusion fluid bag 24 is hung on hook 92 and urine collection chamber or bag 52 is hung on hook 91 via chain 53 and hook 90. Unit 34 includes one or more weight scales such as an electronic strain gage or other means to periodically detect the weight of the collected urine in bag 52 and, if desired, the weight of the remaining hydration fluid in bag 24. Hooks 91 and 92 are connected to a system of levers which translates force to a scale such as a strain gage within unit 34. The strain gage converts force into an electronic signal that can be read by a controller. Suitable electronic devices for accurately measuring the weight of a suspended bag with urine are available from Strain Measurement Devices, 130 Research Parkway, Meriden, Conn., 06450. These devices include electronic and mechanical components necessary to accurately measure and monitor weight of containers with medical fluids such as one or two-liter plastic bags of collected urine. For example, the overload proof single point load cell model S300 and the model S215 load cell from Strain Measurement Devices are particularly suited for scales, weighing bottles or bags in medical instrumentation applications. Options and various specifications and mounting configurations of these devices are available. These low profile single point sensors are intended for limited space applications requiring accurate measurement of full-scale forces of 2, 4, and 12 pounds-force. They can be used with a rigidly mounted platform or to measure tensile or compressive forces. A 10,000Ω wheatstone bridge offers low power consumption for extended battery life in portable products. Other examples of gravimetric scales used to balance medical fluids using a controller controlling the rates of fluid flow from the pumps in response to the weight information can be found in U.S. Pat. Nos. 5,910,252; 4,132,644; 4,204,957; 4,923,598; and 4,728,433 incorporated herein by this reference.

It is understood that there are many ways known in the art of engineering to measure weight and convert it into computer inputs. Regardless of the implementation, the purpose of the weight measurement is to detect the increasing weight of the collected urine in the bag 52 and to adjust the rate of infusion or hydration based on the rate of urine flow by the patient by controlling infusion pump 22.

Unit 34 is also typically equipped with the user interface. The interface allows the user to set (dial in) the two or more parameters of therapy such as the duration of hydration and the desired net fluid balance at the end. The amount of urine which must be output by the patient before balancing begins can also be set. The net fluid balance can be zero if no fluid gain or loss is desired. Display indicators on the console show the current status of therapy: the elapsed time, the net fluid gain or loss, the amount of fluid infused, the amount of fluid loss, the loss rate, and/or the infusion rate.

The user interface may also include alarms. The alarms notify the user of therapy events such as an empty fluid bag or a full collection bag as detected by the weight scale. In one proposed embodiment, the urine is collected by gravity. If urine collection unexpectedly stops for any reason, the system will reduce and, if necessary, stop the IV infusion of fluid and alarm the user. Alternatively, the console can include the second (urine) pump similar to infusion pump 22. This configuration has an advantage of not depending on the bag height for drainage and the capability to automatically flush the catheter if it is occluded by temporarily reversing the pump flow direction.

Infusion pump 22 pumps infusion fluid from bag 24 into the patient and is controlled by the controller electronics within the unit which monitors the weight of the urine in urine collection bag 52. In this way, the patient is properly hydrated and the infusion rate of infusion pump 22 is automatically adjusted to achieve, as necessary, a zero, positive, or negative net fluid balance in the patient.

The electronic controller may also incorporate a more advanced feature allowing the physician to set a desired (for example positive) hydration net goal. For example, the physician may set the controller to achieve positive or negative net gain of 400 ml in 4 hours. The controller calculates the trajectory and adjusts the infusion pump flow rate setting to exceed the urine output accordingly. For example, to achieve a positive net gain of 400 ml over 4 hour, the controller may infuse 25 ml of hydration fluid every 15 minutes in addition to the volume of urine made by the patient in each 15 minute interval. See also co-pending U.S. application Ser. Nos. 11/408,391; 11/408,851; and 11/409,171 filed Apr. 21, 2006 which are incorporated herein by this reference.

In accordance with one example, the infusion set includes infusion bag “spike” connector 20 received in infusion fluid bag 24, luer connector 28 for receiving an IV needle, and tubing extending therebetween and placed within infusion pump 22. The urine collection set typically includes urine collection bag 52, Foley catheter connector 26 for connection to a Foley catheter, and tubing extending between the urine collection bag and connector 26. The infusion set and the urine collection set are preferably placed together as a kit for the hydration unit in sealed bag for storage in a sterile fashion until ready for use. The integrated infusion set includes an IV bag spike, a Luer-to-Foley connector for priming, and a urine collection set includes an integrated urine bag.

The power requirements are typically 115/220 VAC, 60/50 Hz, 25 VA. An auxiliary ground post (potential equalization) for the device is on the rear of the case (not shown). An RS 232 port is also provided. When mounted on an I.V. Pole, the system requires an area of approximately 20×20 inches. Console 34 is placed on the pole so that the urine collection bag 504 is above floor level and not touching the floor or other equipment. Urine collection bag chain 53 is passed through motion restrictor ring 60 to prevent excessive swinging of the bag. Urine collection bag 52 is below the level of patient to facilitate urine drainage, and urine 52 and hydration fluid 24 bags are hanging freely on hooks 90 and 92, respectively, and not supported or impeded. Protection tubes 94 and 96 shown in phantom may be provided about hooks 91 and 92.

The system maintains hydration balance by measuring patient urine output and infusing hydration fluid (prescribed by physician) into the patient I.V. to balance the fluid lost in urine. In addition to urine volume replacement, the system implements a user-set net fluid gain or loss. Net fluid gain is defined as the amount of fluid in ml/hour infused into I.V. in addition to the replaced volume of urine. The system also allows rapid infusion of a Bolus of fluid at the user request. The amount of Bolus can be selected by user and typically the bolus is infused over 30 minutes. Bolus is infused in addition to the Net Fluid Gain and the replaced volume of urine. Unit 34 typically includes a microcontroller device that has means for measuring urine output and the ability to infuse hydration fluid into the patient. The infusion set allows the console to pump fluid from a hydration fluid bag to the patient at a controlled rate. The disposable urine collection set collects the patient's urine to allow it to be measured accurately. Unit 34 is also equipped with an internal battery that can sustain operation in the event of power outage or during short periods of time, for example, when the patient is moved. Unit 34 may include roller pump 22, a user interface, two weighing scales (not shown), air detector 70, post-pump pressure sensor 72, an electrical connector for AC power, and mechanical interfaces for holding the set in place. Console 34 controls the rate at which fluid is infused and monitors urine volume by weight measurement.

Also shown in FIG. 1 is fluid infusion measurement device 10. This device is a component of another infusion subsystem for infusing fluid from bag 12 (a drug, saline, or sodium bicarbonate, for example) into the patient via W needle 13 connected to bag 12 by tubing 14. Fluid infusion measurement device 10 measures the amount of fluid from bag 12 infused into the patient. In one example, device 10 is connected to unit 34 via line 15 and unit 34 displays the output of device 10 (the amount of fluid infused from source 12, and/or the infusion rate, or the weight of source 12 at any given time). Wireless communications between device 10 and unit 34 are also possible. Device 10 could also be integrated into unit 34. Now the nurse will be able to determine, on the display, the amount of saline infused from bag 24, the amount of fluid infused from bag 12, and the amount of urine output by the patient, among other possible readings. Additional sources of infused fluids can be monitored in the same manner.

In one particular example, device 10 includes housing 16 suspended from IV pole 84 via attachment hook 17. Housing 16 also includes attachment hook 18 for suspending fluid bag 12 from housing 16. When device 10 operates on the principle of the weight of bag 12, its output can be a weight value or device 10 can include processing electronics which converts weight into a fluid amount quantity. Housing 10 may also include a display 19 for displaying the fluid amount infused for use and operation independent of unit 34. Other indicators such as a lamp or alarm for indicating when bag 12 is empty, for example, are also possible. A button 20 can be present to reset unit 10 when bag 12 is replaced with a new bag.

Also possible in the system of this invention is regulator 21 controlled by unit 34 via line 23. Wireless operation between unit 34 and regulator 21 is also possible. Unit 34 controls regulator 21 to vary the infusion rate of fluid from bag 12 into the patient. Regulator 21 may include an electrically operatable valve or a pump. The controller of unit 34 can be configured to adjust regulator 21 based on a number of criteria: the amount of hydration fluid infused into the patient from source 24, the amount of urine output by the patient, and/or the mount of fluid from source 12 received by the patient. Another possible criteria is the patient's hydration state. See co-pending application Ser. No. 11/409,171 incorporated herein by this reference. In such an example, a hydration sensor provides an output to unit 34 and the controller thereof is configured to control pump 22 and/or regulator 21 accordingly.

Also, if fluid balancing is desired, the controlling electronics of unit 34 can be configured to adjust the operation of pump 22 based on the amount of fluid received by the patient from source 12, the amount of fluid received by the patient from source 24, and the amount of fluid (urine) output by the patient. In this example, regulator 21 controlled by unit 34 is optional. For example, assume fluid balancing is maintained for a time period during which the patient's urine output is 1 liter per hour. During this time period, pump 22 is set to deliver hydration fluid from source 24 to the patient at a rate of 1 liter per hour. Then, the patient is also infused with fluid from source 12 at a fixed rate of ½ liter per hour. At this time, device 10 provides as an input to unit 34 an indication that the patient is now receiving 12 liter per hour of fluid from source 12. The controlling electronics of unit 34 now controls pump 22 to only deliver ½ liter per hour from source 24 so that the total fluid input to the patient is still 1 liter per hour to balance the urine output by the patient at a rate of 1 liter per hour.

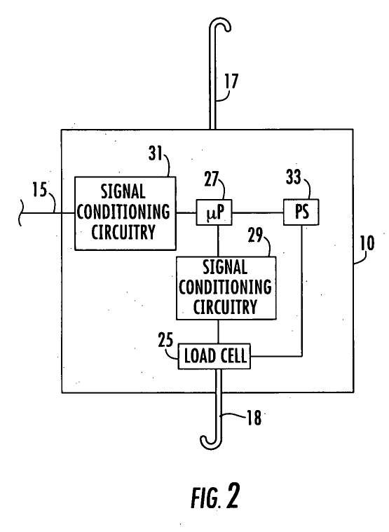

FIG. 2 shows an example of fluid infusion measurement device 10. Load cell 25 is operable to weigh a source of fluid placed on attachment hook 18. The output of load cell 25 is provided to processor 27 typically after conditioning by signal conditioning circuitry 29. Processor 27 is preferably programmed to calculate the amount of fluid in the source of fluid and to track that amount to derive the amount of fluid which has been infused into the patient. That output is provided as shown at 15 typically after conditioning by signal conditioning circuitry 31. The output can be provided to unit 34, FIG. 1 and/or to a display associated with device 10. Power supply 33 may be a battery with its associated power supply circuitry or when device 10 is powered by an outside AC source, power supply 33 is the appropriate power supply circuitry for that source. Note, however, that the processing/controlling electronics of unit 34, FIG. 1 and device 10 may be shared, housed in either unit 34 or device 10, or distributed between the two units. Also, as noted above, load cell 25, FIG. 2 and attachment hook 18 could be integrated within unit 34, FIG. 1.

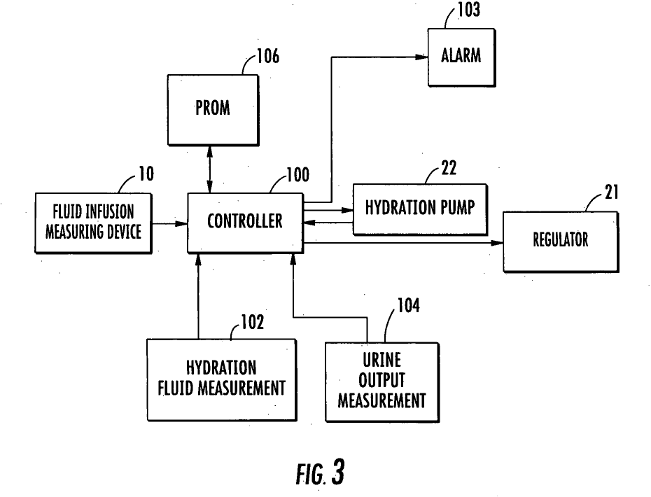

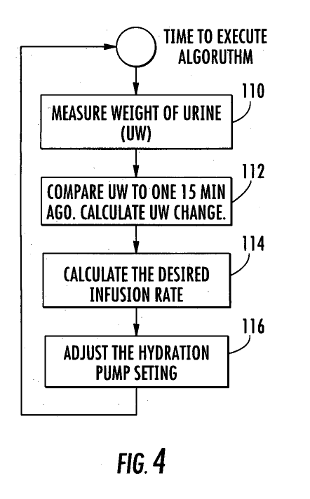

In the subject invention, controller 100, FIG. 3 (a microprocessor or microcontroller or other circuitry (e.g., a comparator) in console 34, FIG. 1 controls hydration pump 22, FIG. 3 to infuse the patient with hydration fluid based on the patient's urine output and keeps track of the hydration fluid injected in two ways to provide safety and redundancy. The preferred hydration fluid measurement subsystem includes, first, as discussed above, the weight of hydration fluid source 24, FIG. 1 which is monitored as shown at 102 in FIG. 3. Urine output is also monitored as shown at 104. In addition, the operation history of infusion pump 22 may be monitored by controller 100. Controller 100 may store values representing both of these measurements in a memory such as PROM 106 and controller 100 is programmed as shown in FIG. 4 to store the hydration fluid amounts administered via the hydration fluid measurement strain gauge, and controller 100 is also programmed to store the hydration fluid amount administered by monitoring of the hydration pump operation history.

Device 10 provides its digital or analog output to controller 100 which, as discussed above, may be distributed between unit 34, FIG. 1 and device 10. Controller 100, FIG. 3, based on the weight measurement, calculates the amount of fluid in source 12, FIG. 1. Controller 100 may also control regulator 21.

Now the operation of controller 100 can take many forms. In the simplest example, the amount of fluid infused from source 12, FIG. 1 is simply displayed on unit 34, FIG. 1. In another example, regulator 21 is controlled based on the amount of urine output by the patient known to the controller as shown at 104. In still another example, regulator 21 is controlled based on the amount of hydration fluid infused into the patient known to controller 100 as shown at 102. Also, the operation of hydration pump 22 can be varied by controller 100 based on the output of fluid infusion measurement device 10.

FIG. 4 illustrates an algorithm that can be used by the controller software of controller 100, FIG. 3 to execute a desired therapy. The algorithm is executed periodically based on a controller internal timer clock. It is appreciated that the algorithm can be made more complex to improve the performance and safety of the device. Controller 100, FIG. 3 is programmed to determine the rate of change of the urine weight, steps 110 and 112, FIG. 4 to calculate a desired infusion rate based on the rate of change of the urine weight, step 114, and to adjust the infusion rate of the infusion pump 22, FIG. 1 based on the calculated desired infusion rate, step 116, FIG. 4.

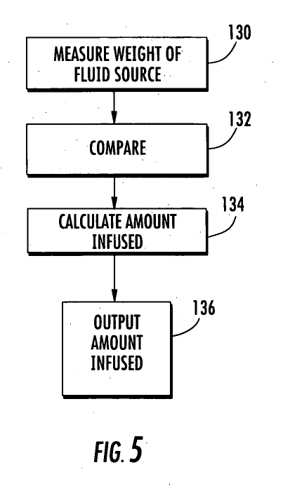

The programming of controller 100 and/or processor 27, FIG. 2 (if present) calculates the amount of fluid infused into the patient from source 12, FIG. 1 by measuring the weight of source 12, step 130, FIG. 5. At different times, the weight of the source is compared, step 132 and based on weight differences, the amount infused is calculated, step 134. The amount infused and/or the infusion rate is then output for display and/or as an input to other programming configured as discussed above when the amount of fluid infused from source 12 is taken into account to control pump 22, to control regulator 21 (if present), and the like.

The result, in any of the various possible embodiments, is a highly versatile fluid management system. When additional fluid sources are added, so too are additional fluid infusion measurement devices. But, as noted above, in other embodiments, fluid infusion measurement device 10, FIG. 1 can be used separately and apart from balancing unit 34. Device 10, for example, may include its own display and/or user interface or may interface with a laptop, personal, or other computer.

Although specific features of the invention are shown in some drawings, then, and not in others, this is for convenience only as each feature may be combined with any or all of the other features in accordance with the invention. For example, there are other ways to determine a patient's urine output and other ways to quantify the amount of hydration fluid administered to the patient. Also, the words “including”, “comprising”, “having”, and “with” as used herein are to be interpreted broadly and comprehensively and are not limited to any physical interconnection. Moreover, any embodiments disclosed in the subject application are not to be taken as the only possible embodiments. Other embodiments will occur to those skilled in the art and are within the following claims.

In addition, any amendment presented during the prosecution of the patent application for this patent is not a disclaimer of any claim element presented in the application as filed: those skilled in the art cannot reasonably be expected to draft a claim that would literally encompass all possible equivalents, many equivalents will be unforeseeable at the time of the amendment and are beyond a fair interpretation of what is to be surrendered (if anything), the rationale underlying the amendment may bear no more than a tangential relation to many equivalents, and/or there are many other reasons the applicant can not be expected to describe certain insubstantial substitutes for any claim element amended.

What is claimed is:

1. A patient hydration system comprising:
a first infusion subsystem for infusing a patient with fluid from a first source;
at least a second infusion subsystem for infusing a patient with fluid from a second source;
a urine output measurement subsystem for determining the amount of urine output by the patient; and
a controller, responsive to the first infusion subsystem, the second infusion subsystem, and the urine output measurement subsystem and configured to control the first infusion subsystem based on the amount of urine output by the patient.

2. The system of claim 1 in which the first infusion subsystem includes a pump controlled by the controller for infusing the patient with fluid from the first source.

3. The system of claim 1 in which the first infusion subsystem further includes a first weighing device for weighing the first source and outputting the weight of the first source to the controller.

4. The system of claim 2 in which the urine output measurement subsystem includes a second weighing device for weighing a urine collection chamber connected to the patient and outputting the weight of the urine collection chamber to the controller.

5. The system of claim 4 in which the controller is programmed to control the pump based on the weight of the urine collection chamber.

6. The system of claim 1 in which the second infusion subsystem includes a weighing device for weighing the second fluid source and outputting the weight of the second fluid source to the controller.

7. The system of claim 6 in which the controller is configured to calculate, based on the weight of the second fluid source, the amount of fluid from the second fluid source infused into the patient and/or the rate of infusion of the fluid from the second source.

8. The system of claim 1 in which the second infusion subsystem includes a regulator for controlling the infusion rate of the fluid from the second source into the patient.

9. The system of claim 8 in which the regulator includes a valve.

10. The system of claim 8 in which the regulator includes a pump.

11. The system of claim 8 in which the controller is configured to adjust the regulator.

12. The system of claim 11 in which the controller is configured to adjust the regulator based on the amount of urine output by the patient.

13. The system of claim 11 in which the controller is configured to adjust the regulator based on the amount of the first fluid infused into the patient from the first source.

14. The system of claim 11 in which the first infusion subsystem includes a pump controlled by the controller for infusing the patient with fluid from the first source, and the controller controls both the pump and the regulator.

15. The system of claim 14 in which the second infusion subsystem includes a second weighing device for weighing the second source and outputting the weight of the second source to the controller.

16. The system of claim 15 in which the controller is configured to control the pump based on the amount of fluid from the second source infused into the patient.

17. The system of claim 1 in which the second infusion subsystem includes a weighing device and a processor responsive to the weighing device for calculating the amount of fluid from the second source infused into the patient based on the weight of the second source.

18. A fluid infusion measurement device comprising:
a housing;
a first attachment for suspending the housing;
a second attachment for suspending a source of fluid from the housing;
a weighing device responsive to the second attachment for weighing the source of fluid infused into a patient; and
a processor responsive to the weighing device and configured to calculate as an output the amount of fluid from the source infused based on the weight of the source.

19. The device of claim 18 in which the housing includes a display for displaying the output of the processor.

20. A patient fluid administration management method comprising:
infusing a patient with a hydration fluid;
administering at least a second fluid to the patient;
measuring the amount of the second fluid administered to the patient;
measuring the patient's urine output; and
controlling the amount of hydration fluid infused into the patient based on the measured urine output.

21. The method of claim 20 further including the step of displaying the measured amount of the second fluid administered to the patient.

22. The method of claim 20 further including the step of controlling the amount of hydration fluid infused into the patient based on the measured amount of the second fluid infused into the patient.

23. The method of claim 20 further including the step of controlling the amount of the second fluid administered to the patient.

24. The method of claim 23 in which the amount of the second fluid administered to the patient is based on the measured urine output.

25. The method of claim 23 in which the amount of the second fluid administered to the patient is based on the amount of hydration fluid infused into the patient.

26. The method of claim 23 in which the amount of the second fluid administered to the patient is based on both the measured urine output and the amount of hydration fluid infused into the patient.

27. A patient hydration system comprising:
a first infusion subsystem for infusing a patient with fluid from a first source;
at least a second infusion subsystem for infusing a patient with fluid from a second source, the second infusion subsystem including:
a housing,
a first attachment for suspending the housing,
a second attachment for suspending the second source from the housing, and
a weighing device responsive to the second attachment for weighing the second source;
a urine output measurement subsystem for determining the amount of urine output by the patient; and
a controller, responsive to the first infusion subsystem, the second infusion subsystem, and the urine output measurement subsystem and configured to control the first infusion subsystem based on the amount of urine output by the patient and to calculate the amount of fluid from the second source infused based on the weight of the second source.
